(19) United States
(12) Patent Application Publication
(10) Pub. No.: US 2009/0242831 A1
(43) Pub. Date: Oct. 1, 2009

(54) AROMATIC HALOSULFONYL ISOCYANATE COMPOSITIONS

(75) Inventors: Gary William Yeager, Rexford, NY (US); Michael Todd Luttrell, Clifton Park, NY (US); Jiang Ji, Clifton Park, NY (US)

Correspondence Address: GENERAL ELECTRIC COMPANY;GLOBAL RESEARCH, PATENT DOCKET RM. BLDG. K1-4A59, NISKAYUNA, NY, 12309, US

(73) Assignee: GENERAL ELECTRIC COMPANY, SCHENECTADY, NY (US)

(21) Appl. No.: 12/056,400

(22) Filed: Mar. 27, 2008

(51) Int. Cl. C07C 265/00; C07C 265/12; C09K 3/00

(52) U.S. Cl. 252/182.3; 560/330; 560/358

(57) ABSTRACT

The present invention provides a monomer composition comprising an aromatic halosulfonyl isocyanate having structure I

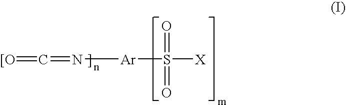

wherein “m” is an integer from 2 to 5; “n” is an integer from 1 to 5; Ar is a C₃-C₄₀ aromatic radical which is free of aliphatic carbon-hydrogen bonds; and X is halogen. The monomer compositions comprising aromatic halosulfonyl isocyanate I are useful in the preparation of polymeric materials useful as membranes.

### BACKGROUND

The invention relates to an aromatic halosulfonyl isocyanate compound and monomer compositions comprising an aromatic halosulfonyl isocyanate compound. Further, the present disclosure relates to a polymer composition derived from the aromatic halosulfonyl isocyanate compound. In addition, the present disclosure relates to a method of using the polymer composition and related articles comprising the polymer composition and includes embodiments that relate to a membrane.

Membranes have a long history of use in separating components of a solution where they are employed as a type of filter able to retain certain substances while transmitting others. The properties and characteristics of membranes depend at least in part on the nature of the material from which the membranes are made. In order to be economically viable, the membrane must provide sufficient flux (the rate of permeate flow per unit of membrane area) and separation (the ability of the membrane to retain certain components while transmitting others). Membranes with high flux and selectivity, and useful levels of hydrophilicity, wetability and chemical resistance find use in applications including ultrafiltration, microfiltration, hyperfiltration, hemofiltration. Fouling of membranes by chemicals, biological compounds, bacteria and cells can negatively impact the flux and selectivity of porous membranes. In applications in which porous membranes are brought into contact with body fluids, immunogenicity and thrombosis are concerns.

Membranes prepared from cellulose acetate also known as semi-permeable membranes show poor performance with respect to hydrolysis, bacterial and chemical attack. While trying to improve their permeability, other properties such as pressure resistance and durability are sacrificed, thereby restricting their application.

In addition to being classified based on their pore size, the membranes can also be classified by their structure, for example as symmetric, asymmetric, and composite membranes. Symmetric membranes are characterized by having a homogeneous pore structure throughout the membrane material. Asymmetric membranes are characterized by a heterogeneous pore structure throughout the membrane material. Composite membranes are defined as having at least one thin film (matrix) layered on a porous support membrane. The porous support membrane can be a polymeric ultrafiltration or microfiltration membrane. The thin film is usually a polymer of a thickness of less than about 1 micron. Composite membranes comprising an ultra-thin membrane, which enhances membrane performance and ease of storage in the dry state offer performances advantages over cellulose membranes that need to be stored in wet conditions. However, these composite membranes often do not exhibit good properties such as high solute rejection against both organic and inorganic materials dissolved in water, high water flux rate, durability such as heat resistance, pressure resistance, and chemical resistance.

Current membrane research has focused on the preparation of membranes for Reverse Osmosis (“RO”), Hyperfiltration (“HF”), Nanofiltration (“NF”), Ultrafiltration (“UF”), Pervaporation (“PV”), Diffusion Dialysis (“DD”), Gas Separation (“GS”) and other membrane separation processes, and employ a variety chemistries in pursuit of optimal membrane performance.

Membranes such as the RO and NF membranes are widely employed as permselective membranes for preferential permeation of certain ionic species for applications such as in the demineralization and softening of water. The type of membrane employed influences the operating conditions chosen for a particular application. For example, a spiral wound RO membrane used in the desalination of seawater generally requires a membrane flux of at least 0.6 cubic meter per day per square meter of membrane at a pressure gradient of about 40-100 atmospheres with a salt rejection of preferably about 99%. In the case of brackish water that has typically about one-tenth the saline concentration of seawater a membrane flux of at least 0.8 meter per day is required at a maximum of about 20 atmospheres pressure gradient and with a salt rejection of about 95%. However, in the case of NF membranes, rejection of ions at minimum pressure gradient and at a flux of at least 0.8 meter per day may be used for desalination of seawater, or brackish water or potable water.

In addition, to be useful in many applications the membranes need to exhibit properties such as high durability, resistance to bioadhesion, microbial adhesion, resistance to oxidants, which may be present in the fluid processed. Further, the membranes should offer resistance to pH fluctuation and fouling by chemicals.

Various approaches have been employed to manufacture thin film composite (“TFC”) permselective membranes using polyamide TFC, RO, and NF membranes. In general, polyamide TFC membranes are prepared using an interfacial polymerization of a diamine and a diacyl chloride. For example interfacially polymerized TFC membranes can be prepared by reacting an aqueous solution of piperazine or 1,3-phenylene diamine and 1,3,5-benzene tricarboxylic acid chloride in a non-polar, volatile, water-immiscible solvent.

Despite major advances in membrane technology, membrane performance degradation is observed to correlate with increased permeate flow through the membrane. Such type performance degradation is also observed when commercial polyamide nanofiltration (NF) and reverse osmosis (RO) membranes are utilized to process strongly acidic feeds. Although initially the performance of such membranes may be sufficient to effect the desired separation, performance rapidly deteriorates, and the membranes lose the ability to retain dissolved metals, such as, cations and/or organic compounds in a short period of time. Polymeric membranes exhibiting stability toward acids are known. However, in certain instances when the polymeric membrane has a porous, lower density morphology, the polymeric membrane can transmit a substantial amounts of dissolved acids and are unable to separate dissolved metal cations and organic compounds effectively.

Therefore, there is a need for improved membranes that have combination of high selectivity, flux and chemical tolerance in addition to being efficient and economical. Further there is a need for new polymer compositions that enable membranes having superior hydrophilicity, and high cross-linking density with improved solute rejection against both inorganic and organic materials, water flux, and mechanical durability.

### BRIEF DESCRIPTION

In one aspect, the present invention provides a polymer composition comprising structural units derived from an aromatic halosulfonyl isocyanate having structure I

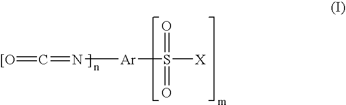

wherein “m” is an integer from 2 to 5; “n” is an integer from 1 to 5; Ar is a C₃-C₄₀ aromatic radical which is free of aliphatic carbon-hydrogen bonds; and X is halogen.

In another aspect, the present invention provides a membrane comprising a polymer composition, wherein the polymer composition comprises structural units derived from an aromatic halosulfonyl isocyanate having structure I

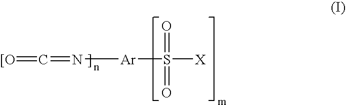

wherein “m” is an integer from 2 to 5; “n” is an integer from 1 to 5; Ar is a C₃-C₄₀ aromatic radical which is free of aliphatic carbon-hydrogen bonds; and X is halogen.

In yet another aspect, the present invention provides a separation unit comprising a plurality of hollow fiber membranes, wherein at least one of the plurality of membranes comprises a membrane formed from a polymer composition comprising structural units derived from an aromatic halosulfonyl isocyanate having structure I

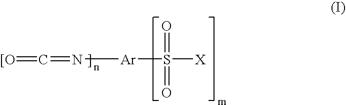

wherein “m” is an integer from 2 to 5; “n” is an integer from 1 to 5; Ar is a C₃-C₄₀ aromatic radical which is free of aliphatic carbon-hydrogen bonds; and X is halogen.

These and other features, aspects, and advantages of the present invention may be understood more readily by reference to the following detailed description.

### DETAILED DESCRIPTION

In the following specification and the claims, which follow, reference will be made to a number of terms, which shall be defined to have the following meanings.

The singular forms “a”, “an” and “the” include plural referents unless the context clearly dictates otherwise.

“Optional” or “optionally” means that the subsequently described event or circumstance may or may not occur, and that the description includes instances where the event occurs and instances where it does not.

As used herein, the term “solvent” can refer to a single solvent or a mixture of solvents.

Approximating language, as used herein throughout the specification and claims, may be applied to modify any quantitative representation that could permissibly vary without resulting in a change in the basic function to which it is related. Accordingly, a value modified by a term or terms, such as “about”, is not to be limited to the precise value specified. In some instances, the approximating language may correspond to the precision of an instrument for measuring the value.

As used herein, the term “aromatic radical” refers to an array of atoms having a valence of at least one comprising at least one aromatic group. The array of atoms having a valence of at least one comprising at least one aromatic group may include heteroatoms such as nitrogen, sulfur, selenium, silicon and oxygen, or may be composed exclusively of carbon and hydrogen. As used herein, the term “aromatic radical” includes but is not limited to phenyl, pyridyl, furanyl, thienyl, naphthyl, phenylene, and biphenyl radicals. As noted, the aromatic radical contains at least one aromatic group. The aromatic group is invariably a cyclic structure having 4n+2 “delocalized” electrons where “n” is an integer equal to 1 or greater, as illustrated by phenyl groups (n=1), thienyl groups (n=1), furanyl groups (n=1), naphthyl groups (n=2), azulenyl groups (n=2), anthraceneyl groups (n=3) and the like. The aromatic radical may also include nonaromatic components. For example, a benzyl group is an aromatic radical, which comprises a phenyl ring (the aromatic group) and a methylene group (the nonaromatic component). Similarly a tetrahydronaphthyl radical is an aromatic radical comprising an aromatic group (C₆H₃) fused to a nonaromatic component —(CH₂)₄—. For convenience, the term “aromatic radical” is defined herein to encompass a wide range of functional groups such as alkyl groups, alkenyl groups, alkynyl groups, haloalkyl groups, haloaromatic groups, conjugated dienyl groups, alcohol groups, ether groups, aldehyde groups, ketone groups, carboxylic acid groups, acyl groups (for example carboxylic acid derivatives such as esters and amides), amine groups, nitro groups, and the like. For example, the 4-methylphenyl radical is a C₇ aromatic radical comprising a methyl group, the methyl group being a functional group which is an alkyl group. Similarly, the 2-nitrophenyl group is a C₆ aromatic radical comprising a nitro group, the nitro group being a functional group. Aromatic radicals include halogenated aromatic radicals such as 4-trifluoromethylphenyl, hexafluoroisopropylidenebis(4-phen-1-yloxy) (i.e., —OPhC(CF₃)₂PhO—), 4-chloromethylphen-1-yl, 3-trifluorovinyl-2-thienyl, 3-trichloromethylphen-1-yl (i.e., 3-CCl₃Ph-), 4-(3-bromoprop-1-yl)phen-1-yl (i.e., 4-BrCH₂CH₂CH₂Ph-), and the like. Further examples of aromatic radicals include 4-allyloxyphen-1-oxy, 4-aminophen-1-yl (i.e., 4-H₂NPh-), 3-aminocarbonylphen-1-yl (i.e., NH₂COPh-), 4-benzoylphen-1-yl, dicyanomethylidenebis(4-phen-1-yloxy) (i.e., —OPhC(CN)₂PhO—), 3-methylphen-1-yl, methylenebis(4-phen-1-yloxy) (i.e., —OPhCH₂PhO—), 2-ethylphen-1-yl, phenylethenyl, 3-formyl-2-thienyl, 2-hexyl-5-furanyl, hexamethylene-1,6-bis(4-phen-1-yloxy) (i.e., —OPh(CH₂)₆PhO—), 4-hydroxymethylphen-1-yl (i.e., 4-HOCH₂Ph-), 4-mercaptomethylphen-1-yl (i.e., 4-HSCH₂Ph-), 4-methylthiophen-1-yl (i.e., 4-CH₃SPh-), 3-methoxyphen-1-yl, 2-methoxycarbonylphen-1-yloxy (e.g., methyl salicyl), 2-nitromethylphen-1-yl (i.e., 2-NO₂CH₂Ph), 3-trimethylsilylphen-1-yl, 4-t-butyldimethylsilylphenl-1-yl, 4-vinylphen-1-yl, vinylidenebis(phenyl), and the like. The term “a C₃-C₁₀ aromatic radical” includes aromatic radicals containing at least three but no more than 10 carbon atoms. The aromatic radical 1-imidazolyl (C₃H₂N₂—) represents a C₃ aromatic radical. The benzyl radical (C₇H₇—) represents a C₇ aromatic radical.

As used herein the term “cycloaliphatic radical” refers to a radical having a valence of at least one, and comprising an array of atoms which is cyclic but which is not aromatic. As defined herein a “cycloaliphatic radical” does not contain an aromatic group. A “cycloaliphatic radical” may comprise one or more monocyclic components. For example, a cyclohexylmethyl group (C₆H₁₁CH₂—) is a cycloaliphatic radical, which comprises a cyclohexyl ring (the array of atoms which is cyclic but which is not aromatic) and a methylene group (the noncyclic component). The cycloaliphatic radical may include heteroatoms such as nitrogen, sulfur, selenium, silicon and oxygen, or may be composed exclusively of carbon and hydrogen. For convenience, the term “cycloaliphatic radical” is defined herein to encompass a wide range of functional groups such as alkyl groups, alkenyl groups, alkynyl groups, haloalkyl groups, conjugated dienyl groups, alcohol groups, ether groups, aldehyde groups, ketone groups, carboxylic acid groups, acyl groups (for example carboxylic acid derivatives such as esters and amides), amine groups, nitro groups, and the like. For example, the 4-methylcyclopent-1-yl radical is a C₆ cycloaliphatic radical comprising a methyl group, the methyl group being a functional group which is an alkyl group. Similarly, the 2-nitrocyclobut-1-yl radical is a C₄ cycloaliphatic radical comprising a nitro group, the nitro group being a functional group. A cycloaliphatic radical may comprise one or more halogen atoms which may be the same or different. Halogen atoms include, for example; fluorine, chlorine, bromine, and iodine. Cycloaliphatic radicals comprising one or more halogen atoms include 2-trifluoromethylcyclohex-1-yl, 4-bromodifluoromethylcyclooct-1-yl, 2-chlorodifluoromethylcyclohex-1-yl, hexafluoroisopropylidene-2,2-bis(cyclohex-4-yl) (i.e., —C₆H₁₀C(CF₃)₂ C₆H₁₀—), 2-chloromethylcyclohex-1-yl, 3-difluoromethylenecyclohex-1-yl, 4-trichloromethylcyclohex-1-yloxy, 4-bromodichloromethylcyclohex-1-ylthio, 2-bromoethylcyclopent-1-yl, 2-bromopropylcyclohex-1-yloxy (e.g., CH₃CHBrCH₂C₆H₁₀O—), and the like. Further examples of cycloaliphatic radicals include 4-allyloxycyclohex-1-yl, 4-aminocyclohex-1-yl (i.e., H₂C₆H₁₀—), 4-aminocarbonylcyclopent-1-yl (i.e., NH₂COC₅H₈—), 4-acetyloxycyclohex-1-yl, 2,2-dicyanoisopropylidenebis(cyclohex-4-yloxy) (i.e., —OC₆H₁₀C(CN)₂C₆H₁₀O—), 3-methylcyclohex-1-yl, methylenebis(cyclohex-4-yloxy) (i.e., —OC₆H₁₀CH₂C₆H₁₀O—), 1-ethylcyclobut-1-yl, cyclopropylethenyl, 3-formyl-2-terahydrofuranyl, 2-hexyl-5-tetrahydrofuranyl, hexamethylene-1,6-bis(cyclohex-4-yloxy) (i.e., —OC₆H₁₀(CH₂)₆C₆H₁₀O—), 4-hydroxymethylcyclohex-1-yl (i.e., 4-HOCH₂C₆H₁₀—), 4-mercaptomethylcyclohex-1-yl (i.e., 4-HSCH₂C₆H₁₀—), 4-methylthiocyclohex-1-yl (i.e., 4-CH₃SC₆H₁₀—), 4-methoxycyclohex-1-yl, 2-methoxycarbonylcyclohex-1-yloxy (2-CH₃OCOC₆H₁₀O—), 4-nitromethylcyclohex-1-yl (i.e., NO₂CH₂C₆H₁₀—), 3-trimethylsilylcyclohex-1-yl, 2-t-butyldimethylsilylcyclopent-1-yl, 4-trimethoxysilylethylcyclohex-1-yl (e.g., (CH₃O)₃SiCH₂CH₂C₆H₁₀—), 4-vinylcyclohexen-1-yl, vinylidenebis(cyclohexyl), and the like. The term “a C₃-C₁₀ cycloaliphatic radical” includes cycloaliphatic radicals containing at least three but no more than 10 carbon atoms. The cycloaliphatic radical 2-tetrahydrofuranyl (C₄H₇O—) represents a C₄ cycloaliphatic radical. The cyclohexylmethyl radical (C₆H₁₁CH₂—) represents a C₇ cycloaliphatic radical.

As used herein the term “aliphatic radical” refers to an organic radical having a valence of at least one consisting of a linear or branched array of atoms, which is not cyclic. Aliphatic radicals are defined to comprise at least one carbon atom. The array of atoms comprising the aliphatic radical may include heteroatoms such as nitrogen, sulfur, silicon, selenium and oxygen or may be composed exclusively of carbon and hydrogen. For convenience, the term “aliphatic radical” is defined herein to encompass, as part of the “linear or branched array of atoms which is not cyclic” a wide range of functional groups such as alkyl groups, alkenyl groups, alkynyl groups, haloalkyl groups, conjugated dienyl groups, alcohol groups, ether groups, aldehyde groups, ketone groups, carboxylic acid groups, acyl groups (for example carboxylic acid derivatives such as esters and amides), amine groups, nitro groups, and the like. For example, the 4-methylpent-1-yl radical is a C₆ aliphatic radical comprising a methyl group, the methyl group being a functional group which is an alkyl group. Similarly, the 4-nitrobut-1-yl group is a C₄ aliphatic radical comprising a nitro group, the nitro group being a functional group. An aliphatic radical may be a haloalkyl group which comprises one or more halogen atoms which may be the same or different. Halogen atoms include, for example; fluorine, chlorine, bromine, and iodine. Aliphatic radicals comprising one or more halogen atoms include the alkyl halides trifluoromethyl, bromodifluoromethyl, chlorodifluoromethyl, hexafluoroisopropylidene, chloromethyl, difluorovinylidene, trichloromethyl, bromodichloromethyl, bromoethyl, 2-bromotrimethylene (e.g., —CH₂CHBrCH₂—), and the like. Further examples of aliphatic radicals include allyl, aminocarbonyl (i.e., —CONH₂), carbonyl, 2,2-dicyanoisopropylidene (i.e., —CH₂C(CN)₂CH₂—), methyl (i.e., —CH₃), methylene (i.e., —CH₂—), ethyl, ethylene, formyl (i.e., —CHO), hexyl, hexamethylene, hydroxymethyl (i.e., —CH₂OH), mercaptomethyl (i.e., —CH₂SH), methylthio (i.e., —SCH₃), methylthiomethyl (i.e., —CH₂SCH₃), methoxy, methoxycarbonyl (i.e., CH₃OCO—), nitromethyl (i.e., —CH₂NO₂), thiocarbonyl, trimethylsilyl (i.e., (CH₃)₃Si—), t-butyldimethylsilyl, 3-trimethyoxysilylpropyl (i.e., (CH₃O)₃SiCH₂CH₂CH₂—), vinyl, vinylidene, and the like. By way of further example, a C₁-C₁₀ aliphatic radical contains at least one but no more than 10 carbon atoms. A methyl group (i.e., CH₃—) is an example of a C₁ aliphatic radical. A decyl group (i.e., CH₃(CH₂)₉—) is an example of a C₁₀ aliphatic radical.

As noted, in one embodiment the present invention provides a monomer composition comprising an aromatic halosulfonyl isocyanate having structure I

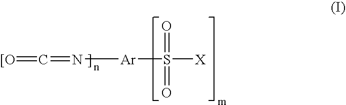

wherein “m” is an integer from 2 to 5; “n” is an integer from 1 to 5; Ar is a C₃-C₄₀ aromatic radical which is free of aliphatic carbon-hydrogen bonds; and X is halogen. In one embodiment, “m” is 2. In another embodiment, “m” is 3. In yet another embodiment, “n” is 1 and in another embodiment, “n” is 2.

Representative aromatic halosulfonyl isocyanates encompassed by generic structure I are illustrated in Table 1. One of ordinary skill in the art will appreciate the relationship between generic structure I and the individual structures of Entries 1a-1 h of Table 1. For example, the structure of Entry 1a represents a species encompassed by generic structure I wherein, Ar is a C₆ aromatic ring (a benzene ring), the variable “n” is 1, “m” is 3, X is chloride.

TABLE 1

| Entry Number | Structure |
|---|---|
| 1a | 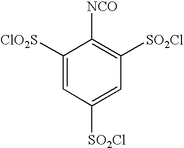 |
| 1b | 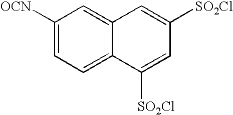 |
| 1c | 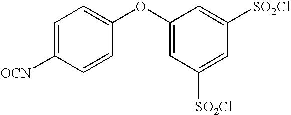 |
| 1d | 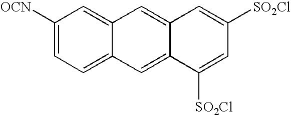 |
| 1e | 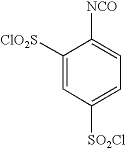 |

By way of further example, Entry 1b of Table 1 illustrates an aromatic halosulfonyl isocyanate wherein Ar is napthalene, “n” is 1, “m” is 2, and X is chloride. Entry 1c of Table 1 illustrates an aromatic halosulfonyl isocyanate wherein Ar is phenoxybenzene, “n” is 1, “m” is 2, and X is chloride.

In one embodiment, the present invention provides an aromatic halosulfonyl isocyanate having structure I wherein the group Ar is a C₆-C₂₀ aromatic radical. In some embodiments, the group Ar is a trivalent aromatic radical having structure II.

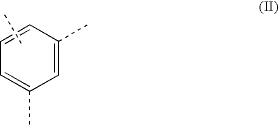

For example, the structure of Entry 1e represents a species encompassed by generic structure I wherein, Ar has structure II, i.e. a trisubstituted phenyl ring wherein at least two of the substituents are located at ring positions which are “meta” to one another.

In one embodiment, the present invention provides an aromatic halosulfonyl isocyanate having structure I, wherein the group Ar is a trivalent aromatic radical having structure III

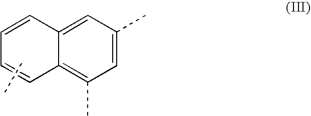

By way of example, Entry 1b of Table 1 illustrates an aromatic halosulfonyl isocyanate wherein Ar is a trisubstituted naphthalene ring.

In another embodiment, the aromatic halosulfonyl isocyanate composition provided by the present invention has a structure IV.

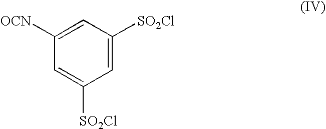

And in yet another embodiment, the aromatic halosulfonyl isocyanate provided by the present invention has a structure V.

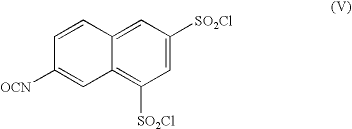

In one embodiment, the aromatic halosulfonyl isocyanate composition further comprises a C₃-C₄₀ aromatic monomer having a functionality of at least two. In another embodiment, the aromatic halosulfonyl isocyanate composition further comprises a C₃-C₄₀ aromatic monomer having a functionality of two. As used herein the expression “having a functionality of two” means that aromatic monomer contains one halsulfonyl (SO₂X) group and one isocyanato (NCO) group. In one embodiment, the halosulfonyl isocyanate having a functionality of two has structure VI

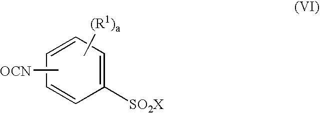

wherein R¹ is independently at each occurrence, a hydrogen atom, a halogen atom, a nitro group, a cyano group, a C₁-C₂₀ aliphatic radical, a C₃-C₂₀ cycloaliphatic radical, or a C₃-C₂₀ aromatic radical; “a” is an integer from 1 to 4; and X is halogen.

In one embodiment, the present invention provides a halosulfonyl isocyanate composition comprising structure I together with a halosulfonyl isocyanate having a functionality of two having a structure VI wherein X is chlorine. In one embodiment, R¹ of structure VI is an electrophilic group, for example a chlorocarbonyl group. As defined herein the chlorocarbonyl group represents a C₁ aliphatic radical (COCl). Additional non limiting examples of the group R¹ include a carbonyl halide, an alpha haloketo group, a haloformate, an acid anhydride, a phosphorylhalide, a glycidyl ether.

In another embodiment, the present invention provides a halosulfonyl isocyanate composition comprising an aromatic halosulfonyl isocyanate having structure I and a second aromatic halosulfonyl isocyanate having a functionality of two and having structure VII

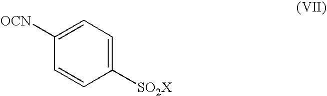

wherein X is halogen.

In yet another embodiment, the present invention provides a halosulfonyl isocyanate composition comprising an aromatic halosulfonyl isocyanate having structure I and a second aromatic halosulfonyl isocyanate having a functionality of two and having structure VIII.

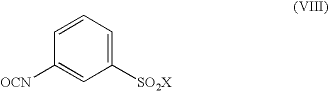

wherein X is halogen.

In another aspect the present invention provides a polymer composition comprising structural units derived from an aromatic halosulfonyl isocyanate having structure I

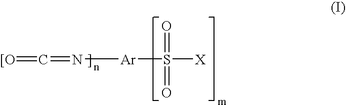

wherein “m” is an integer from 2 to 5; “n” is an integer from 1 to 5; Ar is a C₃-C₄₀ aromatic radical, which is free of aliphatic carbon-hydrogen bonds; and X is halogen. Polymer compositions comprising structural units derived from an aromatic halosulfonyl isocyanate having structure I are illustrated by the polysulfonamide-polyurea polymer prepared by the polymerization of piperazine with monomer V, the polysulfonamide-polyurea polymer prepared by the polymerization of piperazine with monomer 1c (Table 1), the polysulfonamide-polyurea polymer prepared by the polymerization of piperazine with monomer 1d (Table 1). In one embodiment, the polymer compositions comprising structural units derived from an aromatic halosulfonyl isocyanate having structure I further comprise structural units derived from a C₃-C₄₀ aromatic monomer having a functionality of at least two, for example a polymer composition prepared by reacting a halosulfonyl isocyanate composition comprising halosulfonyl isocyanate V and halosulfonyl isocyanate VII. In another embodiment, the polymer provided by the present invention comprises structural units derived from halosulfonyl isocyanate I and structural units derived from at least one additional electrophilic monomer, for example terephthaloyl chloride, toluene diisocyanate, trimellitic anhydride acid chloride, 5-isocyanato isophthaloyl chloride, 5-chloroformyloxy isophthaloyl chloride, 5-chlorosulfonyl isophthaloyl chloride, isophthaloyl chloride and trimesoyl chloride and combinations thereof.

In one embodiment, the present invention provides a polymer composition comprising structural units derived from a halosulfonyl isocyanate having structure I and structural units derived from at least one additional electrophilic monomer selected from the group consisting of isophthaloyl chloride, terephthaloyl chloride, trimesoyl chloride, trimellitic acid trichloride, 1,3-cyclohexane dicarboxylic acid chloride, 1,4-cyclohexane dicarboxylic acid chloride, cyclohexane tricarboxylic acid halides, quinolinic acid dichloride, dipicolinic acid dichloride, trimellitic anhydride acid halides, pyromellitic acid tetra chloride, pyromellitic acid dianhydride, pyridine tricarboxylic acid halides, sebacic acid halides, azelaic acid halides, adipic acid halides, dodecanedioc acid halides, toluene diisocyanate, methylenebis(phenylisocyanate), naphthalene diisocyanates, bitolyl diisocyanates, hexamethylene diisocyanate, phenylene diisocyanates, isocyanatobenzene dicarboxylic acid halides, haloformyloxy benzene dicarboxylic acid halides, dihalosulfonyl benzenes, halosulfonyl benzene dicarboxylic acid halides, cyclobutane dicarboxylic acid halide, piperazine —N—N′-diformyl halides, dimethyl piperazine —N, N′-diformyl halides, xylylene glycol dihaloformates, benzene diol di-haloformates, benzene triol trihaloformates, phosgene, diphosgene, triphosgene, N,N′-carbonyl diimidazole, isocyanuric acid N,N′,N″-triacetyl halide, isocyanuric acid-N,N′,N″ tripropionyl halide, cyclopentane tetracarboxylic acid halides, and combinations thereof.

In one embodiment, the present invention provides a polymer composition comprising structural units derived from a halosulfonyl isocyanate having structure I and structural units derived from an acid halide-terminated oligomer. Acid halide-terminated oligomers are illustrated by the product of reacting piperazine with an excess one or more of isophthaloyl chloride, isophthaloyl chloride, terephthaloyl chloride); trimesoyl chloride, trimellitic acid trichloride, quinolinic acid dichloride, dipicolinic acid dichloride, trimellitic anhydride acid halides, pyromellitic acid tetra chloride, pyromellitic acid dianhydride, pyridine tricarboxylic acid halides, toluene diisocyanate, methylenebis(phenylisocyanates), naphthalene diisocyanates, bitolyl diisocyanates, phenylene diisocyanates, isocyanatobenzene dicarboxylic acid halides, haloformyloxy benzene dicarboxylic acid halides, dihalosulfonyl benzenes, halosulfonyl benzene dicarboxylic acid halides, xylylene glycol dihaloformates, benzene diol di-haloformates, benzene triol trihaloformates, phosgene, diphosgene, triphosgene, and N,N′-carbonyl diimidazole.

The polymer compositions provided by the present invention comprise at least one ureido NH group per structural unit arising from aromatic halosulfonyl isocyanate I. It is believed that the presence of the ureido NH groups provides for enhanced interaction of the polymer composition with aqueous liquids, and provides an additional level of structural integrity in articles comprising the polymer compositions of the present invention through hydrogen bonding between the uriedo NH groups and groups derived from the halosulfonate groups, for example sulfonamide groups. The presence of the ureido NH groups is believed to be of particular importance in embodiments in which the polymer composition is prepared using one or more diamines comprising only secondary amine groups, as in for example piperazine.

In one embodiment, the polymer composition provided by the present invention comprises structural units derived from the aromatic halosulfonyl isocyanate having structure I and structural units derived from a polyamine compound having structure IX

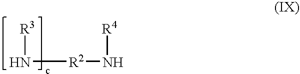

wherein R² is a C₁-C₂₀ aliphatic radical, a C₃-C₂₀ cycloaliphatic radical, or a C₃-C₂₀ aromatic radical; R³ and R⁴ are independently at each occurrence a hydrogen atom, a C₁-C₂₀ aliphatic radical, a C₃-C₂₀ cycloaliphatic radical, or a C₃-C₂₀ aromatic radical, and “c” is an integer from 1 to 10. Structure IX includes instances in which R² may together with R³ and R⁴ form a cyclic structure, for example when structure IX represents the C₄-diamine piperazine wherein “c” is 1, R² is —CH₂CH₂—, and R³ and R⁴ are each —CH₂—, and R³ is linked to R⁴ via a single carbon-carbon bond.

In one embodiment, the polyamine compound having structure IX may contain two amino groups per molecule (i.e “c” is 1). Non-limiting examples of polyamine compounds encompassed by generic structure IX include polyethylenamines, ethylene diamine, diethylene diamine or piperazine, phenylene diamine, meta-phenylene diamine, para-phenylene diamine, cyclohexanediamines, cyclohexanetriamines, xylylenediamines, chlorophenylene diamines, benzenetriamines, bis(aminobenzyl)aniline, tetraminobenzenes, tetraminobiphenyls, tetrakis(aminomethyl)methane, N,N′-diphenyl ethylenediamine, aminobenzamides, aminobenzhydrazides, bis(aminobenzyl)anilines, N,N′-dialkyl-1,3-phenylenediamine, N-alkyl-1,3-phenylenediamine, melamine. In one embodiment, the polyamine having structure IX is 1,3,5-triaminobenzene, piperazine, 4-aminomethylpiperidine, 1,4-phenylene diamine, 1,3-phenylene diamine or a combination of two or more of the foregoing polyamine compounds.

In one embodiment, suitable molecular weights of the polymer composition of the present invention is greater than about 1,000 g/mol. In some embodiments, the molecular weight of the composition is less than about 200,000 g/mol. In one embodiment, suitable molecular weights of the polymer composition of the present invention is in a range from about 1,000 g/mol to about 200,000 g/mol. In one embodiment, the molecular weight of the polymer composition is in a range of from about 1,000 to about 40,000 g/mol, from about 40,000 to about 80,000 g/mol, from about 80,000 to about 120,000 g/mol, or from about 120,000 g/mol to about 200,000 g/mol. In one embodiment, the polymer composition is a copolymer comprising structural units derived from an aromatic halosulfonyl isocyanate having structure I, and a C₃-C₄₀ aromatic monomer having a functionality of two. In various embodiments, the polymer composition provided by the present invention is a homopolymer, a random copolymer, a block copolymer, or a graft copolymer.

In one embodiment, the polymer composition contains one or more additives. The additives may be selected to affect the chacteristics and properties of an article made from the composition. Mixtures of additives may be used. Such additives may be mixed at a suitable time during the mixing of the components for forming the composition. Exemplary additives include extenders, lubricants, flow modifiers, fillers, fire retardants, pigments, dyes, colorants, UV light stabilizers, anti-oxidants, impact modifiers, heat stabilizers, antidrip agents, plasticizers, mold release agents, nucleating agents, optical brighteners, flame proofing agents, anti-static agents, blowing agents, and the like. If present, the additive may be in a range of from about 0.1 weight percent to about 40 weight percent, based on the total weight of composition.

In certain embodiments, the polymer compositions provided by the present invention is used to form ion exchange membranes. In certain other embodiments, the polymer composition is molded into useful articles by a variety of means, for example injection molding, extrusion molding, rotation molding, foam molding, calendar molding, blow molding, thermoforming, compaction, melt spinning, and the like, to form articles. In one embodiment, the polymer compositions can be pulled or spun into the form of a fiber, a sheet or a film. In another embodiment, the polymer compositions can be pulled or spun into a plurality of fibers that define a membrane. The fibers can be elastic and have relative high mechanical properties. Suitable fibers can be hollow fibers. In one embodiment, fibers is arranged to define a mat or a membrane. Further, the membrane can be supported on a second membrane that is itself not formed from a composition including an embodiment of the invention.

In one embodiment, the polymer composition provided by the present invention is used in a film or sheet, which may be perforate, or porous. In one embodiment, the film or sheet is continuous and impermeable. Suitable sheets and films can have a surface topology on one or both major surfaces. Such topology can include patterned microstructures and/or ridges to increase the available surface area or contact area available. In certain embodiments, the sheet or film can be porous or permeable so that a fluid can pass or flow through it. Such a sheet or film is a type of membrane. The membrane can be rendered permeable by one or more of perforating, stretching, expanding, bubbling, or extracting, for example. Suitable methods of making the membrane include foaming, skiving or casting. In one embodiment, a membrane is formed from woven or non-woven fibers. In one embodiment, a membrane provided by the present invention is formed on the surface of a porous substrate, for example a porous polymeric film.

Numerous techniques are known in the art to prepare membranes. For example, membranes can be formed using a dry-phase separation membrane formation process in which a dissolved polymer is precipitated by evaporation of a sufficient amount of solvent to form a membrane structure; a wet-phase separation membrane formation process in which a dissolved polymer is precipitated by immersion in a non-solvent bath to form a membrane structure; a dry-wet phase separation membrane formation process which is a combination of the dry and the wet-phase formation processes; or a thermally-induced phase-separation membrane formation process in which a dissolved polymer is precipitated or coagulated by controlled cooling to form a membrane structure. Further, the membrane can be subjected to a membrane conditioning process, or to a pretreatment process, prior to the membrane's use in a separation application. Representative processes may include thermal annealing to relieve stresses, and pre-equilibration in a solution similar to the feed stream the membrane will contact.

In one embodiment, the membrane is a three-dimensional matrix, or have a lattice type structure including plurality of nodes interconnected by a plurality of fibrils. Surfaces of the nodes and fibrils can define a plurality of pores in the membrane and can define numerous interconnecting pathways or pores that extend through the membrane from one to another opposite major side surfaces in a tortuous path. In one embodiment, the membrane can define many interconnected pores that fluidly communicate with environments adjacent to the opposite facing major sides of the membrane. The propensity of the material of the membrane to permit a liquid material, for example, an aqueous liquid material, to wet out and pass through pores can be expressed as a function of one or more properties. The properties include the surface energy of the membrane, the surface tension of the liquid material, the relative contact angle between the material of the membrane and the liquid material, the size or effective flow area of pores, and the compatibility of the material of the membrane and the liquid material. The membrane can have a plurality of sub layers. The sub layers may be the same as, or different from, each other. In one aspect, one or more sub layer may include an embodiment of the invention, while another sub layer may provide a property such as, for example, reinforcement, selective filtering, flexibility, support, flow control, and the like. Membranes according to embodiments of the invention have differing dimensions, some selected with reference to application-specific criteria. Each membrane may be formed from a plurality of sheets or films, may be formed from a weave or mat of fibers, may include a non-inventive layer, or may include two or more of the foregoing.

A membrane prepared according to embodiments of the invention can have one or more predetermined properties. Such properties can include one or more of a wetability of a dry-shipped membrane, a wet/dry cycling ability, filtering of polar liquid or solution, flow rate of aqueous liquid or solution, surface electronegativity, flow and/or permanence under low pH conditions, flow and/or permanence under high pH conditions, flow and/or permanence at room temperature conditions, flow and/or permanence at elevated temperature conditions, flow and/or permanence at elevated pressures, transparency to energy of predetermined wavelengths, transparency to acoustic energy, or support for catalytic material. Permeance refers to the ability of the coating material to maintain function in a continuing manner, for example, for more than 1 day or more than one cycle (wet/dry, hot/cold, high/low pH, and the like). In one embodiment, the membrane a resistance to temperature excursions in a range of from about 100 degrees Celsius to about 125 degrees Celsius, for example, in autoclaving operations.

Flow rate of fluid through the membrane can be dependent on one or more factors such as for example may depend on the physical and/or chemical properties of the membrane, the properties of the fluid (e.g., viscosity, pH, solute, and the like), environmental properties (e.g., temperature, pressure, and the like), and the like.

In one embodiment, the membrane is used to filter water. A filtration membrane that passes a flow of water from an aqueous solution of relatively high solute concentration to a solution of relatively low solute concentration in response to a pressure differential across the membrane. Thus, in one embodiment, the membrane is operable to have a liquid or fluid flow through at least a portion of the material in a predetermined direction. The motive force may be osmotic or wicking, or may be driven by one or more of a concentration gradient, pressure gradient, temperature gradient, or the like. In another embodiment, the membrane has a salt rejection percentage of greater than 75 percent. In one embodiment the membrane is a reverse osmosis membrane in the water treatment system. In another embodiment, the membrane blocks a flow of ions therethrough. The ions include metal ions.

Other suitable applications can include liquid filtration, polarity-based chemical separations, pervaporization, gas separation, industrial electrochemistry such as chloralkali production and electrochemical applications, super acid catalysts, or use as a medium in enzyme immobilization.

Microfiltration membranes can filter a suspension of fine particles or colloidal particles with linear dimensions in a range of from about 20 nanometers to about 10,000 nanometers. Ultrafiltration membranes may have pore sizes of less than about 100 nanometers on average, and may retain species in the molecular weight range of from about 300 daltons to about 500,000 daltons. Suitable rejected species include sugars, biomolecules, polymers and colloidal particles. Nanofiltration membranes have received increasing attention in low-pressure water desalination. These membranes are often negatively charged and reject salts through charge repulsion (Donnan exclusion). In addition, organic species with molecular weights in the range of about 200 daltons to about 500 daltons are rejected. Hyperfiltration and reverse osmosis (RO) may use a relatively dense membrane. Such dense membrane may have pores or perforations of sufficient size or chemical activity such that small molecules such as salts and low molecular weight organics are treated differently from water in contact with the membrane surface. Suitable RO membranes according to embodiments of the invention may include high pressure RO membranes for desalination of seawater (5 MPa to about 10 MPa driving pressure0; medium pressure RO for desalination of brackish water (1 MPa to about 5 MPa driving pressure); and nanofiltration or “loose” RO for partial demineralization of water (0.3 MPa to about 1 MPa driving pressure, 0-20% NaCl rejection). Both ultrafiltration and microfiltration membranes have been used as interlayer supports in thin film composite membranes. These membranes may be used for numerous water purifications, most notably nano-filtration, reverse osmosis, thin film membrane, and hyperfiltration.

In one embodiment, the invention provides a composite membrane comprising the polymer composition of the present invention located on at least one side of a porous support material. The term “composite membrane” means a composite of a matrix layered or coated on at least one side of a porous support material. The term “support material” means any substrate onto which the matrix can be applied. Included are semipermeable membranes especially of the micro- and ultrafiltration kind, fabric, filtration materials as well as others. In one embodiment, the porous support material can be composed of any suitable porous material including but not limited to paper, modified cellulose, woven glass fibers, porous or woven sheets of polymeric fibers. The porous support materials may comprise a polymer, for example polysulfone, polyethersulfone, polyacrylonitrile, cellulose ester, polyolefin, polyester, polyurethane, polyamide, polycarbonate, polyether, polyarylether ketones, polypropylene, polybenzene sulfone, polyvinylchloride, polyvinylidenefluoride, and combinations thereof, a ceramic membrane; a porous glass; a porous metals; or a combination of two or more of the foregoing polymers, glasses, and metals. The composite membrane may be formed as sheets, hollow tubes, thin films, or flat or spiral membrane filtration devices. In another embodiment, the support materials is polysulfones, polyethersulfones, sulfonated polysulfone, sulfonated polyethersulfone, polyvinylidene fluoride, polytetrafluoroethylene, polyvinyl chloride, polystyrenes, polycarbonates, polyacrylonitriles, polyaramides, nylons, polyamides, polyimides, melamines, thermosetting polymers, polyether ketones, polyetheretherketones), polyphenylenesulfide. In one embodiment, the porous support material is selected from the group consisting of a polyolefin, a polysulfone, a polyether, a polysulfonamide, a polyamine, a polysulfide, a melamine polymer, and combinations thereof.

In one embodiment, the present invention provides a desalination unit comprising the water treatment system comprising the membrane derived from the aromatic halosulfonyl isocyanate compound of the present invention. In another embodiment, the present invention provides an ultrafiltration membrane derived from the aromatic halosulfonyl isocyanate compound of the present invention. In another embodiment, the present invention provides a bioseparation apparatus comprising the membrane that can separate one biological fluid component from another biological fluid component.

In another aspect the invention provides a separation unit comprising a plurality of hollow fiber membranes, wherein at least one of the plurality of membranes comprises a membrane formed from a polymer composition comprising structural units derived from an aromatic halosulfonyl isocyanate having structure I

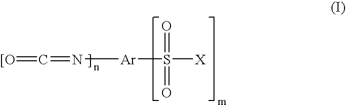

wherein “m” is an integer from 2 to 5; “n” is an integer from 1 to 5; Ar is a C₃-C₄₀ aromatic radical which is free of aliphatic carbon-hydrogen bonds; and X is halogen.

The aromatic halosulfonyl isocyanate compounds and the polymer compositions derived from the halosulfonyl isocyanate compounds of the present invention may be prepared by a variety of methods including those provided in the experimental section of this disclosure.

### EXAMPLES

All materials were obtained from Aldrich Chemical Company. ¹H-NMR was performed on a 400 MHz Bruker NMR spectrometer.

### Example 1 Preparation of 2,4-Bis(chlororsulfonyl)-6-isocyanatonapthalene 1

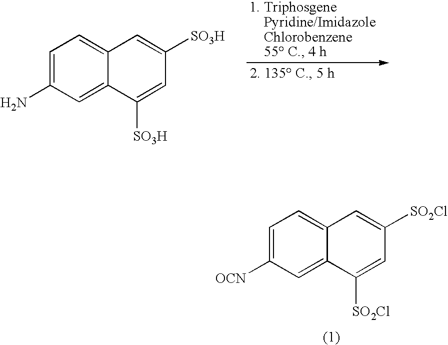

A 500 mL round bottom flask equipped with a magnetic stirrer, nitrogen bubbler, nitrogen inlet and a temperature probe was charged triphosgene (19.57 g, 65.94 mmol), 2-aminonaphthalene-6,8-disulfonic acid (10.0 g, 32.96 mmol) and anhydrous chlorobenzene (100 mL) and cooled with a cooling bath. A separate solution of pyridine:imidazole catalyst (0.625 g pyridine: 0.125 g imidazole) in anhydrous chlorobenzene (50 mL) was added slowly over 15 min. The reaction was then stirred at 5-10° C. for an additional 30 minutes. After this time, the temperature was increased to 55° C. for 4 hours, and further increased to 135° C. for an additional 5 hours. The mixture was then cooled to ambient temperature and concentrated under reduced pressure to afford bischlorosulfonyl isocyanate compound I as a yellow solid.

¹H NMR (CDCl₃): 8.90 (m, 1H); 8.88 (d, J=2.0, 1H); 8.54 (d, J=1.8, 1H); 8.25 (d, J=8.8, 1H); 7.66 (dd, J=8.9, 2.0, 1H).

### Example 2 Preparation of a Microporous Membrane Comprising Structural Units Derived From Bischlorosulfonyl Isocyanate Compound 1

An experimental microporous polyethersulfone ultrafiltration membrane (the “support”) was immersed in an aqueous solution of comprising two weight percent piperazine and 0.1 weight percent N,N-dimethylaminopyridine in water for one minute at room temperature. The support was removed and wiped clean of any residual water droplets to provide a microporous polyethersulfone support impregnated with the aqueous piperazine solution. Other commercially available microporous ultrafiltration membranes, for example the P-Series family of polyethersulfone ultrafiltration membranes available from GE Water, Trevose Pa., may be employed as the support as well.

A solution of bischlorosulfonyl isocyanate compound 1 in ISOPAR-G was heated to approximately 100° C., then poured onto the surface of the impregnated support. Contact between the solution of the bischlorosulfonyl isocyanate compound 1 and the impregnated support was maintained for 2 minutes during which time the temperature of the organic solution decreased to approximately 40° C. The organic solution was decanted from the support and the treated support was cured in an oven at 120° C. for 6 minutes and then cooled to room temperature to provide a microporous membrane comprising the microporous polyethersulfone support coated with a polysulfonamide-polyurea comprising structural units derived from bischlorosulfonyl isocyanate 1 and piperazine.

### Example 3 Evaluation of Membrane of Prepared in Example 2

Test coupons (5″×3″) were cut from the microporous membrane prepared in Example 2 and were fixed in a cross-flow cell membrane testing bench. The test coupons were treated with a salt solution containing 2000 ppm NaCl in dionized water at 800 psi and 20° C. for 1 hour. After this time, the permeate from each replicate was collected over a recorded time, the volume collected was determined, and the permeate conductivity was measured (in μS) using an Oakton Acorn CON 6 conductivity meter to obtain the percent salt passage. The membrane permeability (A-value) was calculated from data including the pressure, the area of the membrane and the recorded time and permeate volume. While the membrane remained in the test apparatus, the membrane was treated with an aqueous solution of sodium hypochlorite (70 ppm) in deionized water at 225 psi and 20° C. for 30 minutes. Following treatment with the sodium hypochlorite solution, the membrane was rinsed with deionized water for 30 minutes, and then treated with a salt solution containing 2000 ppm NaCl in dionized water at 800 psi and 20° C. for 1 hour. The permeate was collected over a recorded period of time, the volume of the permeate was determined over this time, and the conductivity of the permeate was measured as before to obtain percent salt passage of the membrane following treatment sodium hypochlorite solution. The membrane permeability (A-value) was again calculated.

The data reveal that the microporous membrane prepared in Example 2 functions effectively as a reverse osmosis membrane and are gathered in Table 2 below. The data show that the membrane performance is not degraded by treatment with sodium hypochlorite in a regeneration step. The data in Comparative Example 1 (CE-1) further illustrate that the membrane of Example 3 performs at least as well as a known microporous membrane prepared identically to that used in Example 3 and comprising structural units derived from piperazine and of 2,4,6-tis(chlororsulfonyl)-napthalene. Those of ordinary skill in the art will recognize that the microporous membrane of Comparative Example 1 lacks any urido NH groups, a structural feature believed to enhance the overall performance of the microporous membrane provided by the present invention.

TABLE 2

| | Example 3 | CE-1 |
|---|---|---|
| Thin Film Composite Membrane | | |
| Properties | | |
| A-Value (g/(h * cm² * Pa)^{a} | 6.9 | 7.5 |
| Standard Deviation | 2.9 | 3.8 |
| NaCl Rejection %^{a} | 66.5 | 70.7 |
| Standard Deviation | 15.7 | 7.5 |
| Thin Film Composite Membrane | | |
| Properties after Chlorine | | |
| Post-Treatment | | |
| A-Value (g/(h * cm2 * Pa)^{a} | 5.9 | 6.2 |
| Standard Deviation | 1.9 | 2.6 |
| NaCl Rejection %^{a} | 67.7 | 76.6 |
| Standard Deviation | 20 | 21.7 |

The foregoing examples are merely illustrative, serving to illustrate only some of the features of the invention. The appended claims are intended to claim the invention as broadly as it has been conceived and the examples herein presented are illustrative of selected embodiments from a manifold of all possible embodiments. Accordingly, it is the Applicants' intention that the appended claims are not to be limited by the choice of examples utilized to illustrate features of the present invention. As used in the claims, the word “comprises” and its grammatical variants logically also subtend and include phrases of varying and differing extent such as for example, but not limited thereto, “consisting essentially of” and “consisting of.” Where necessary, ranges have been supplied; those ranges are inclusive of all sub-ranges there between. It is to be expected that variations in these ranges will suggest themselves to a practitioner having ordinary skill in the art and where not already dedicated to the public, those variations should where possible be construed to be covered by the appended claims. It is also anticipated that advances in science and technology will make equivalents and substitutions possible that are not now contemplated by reason of the imprecision of language and these variations should also be construed where possible to be covered by the appended claims.

What is claimed is:

1. A monomer composition comprising an aromatic halosulfonyl isocyanate having structure I 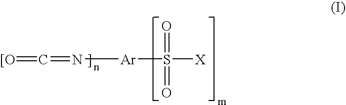
wherein “m” is an integer from 2 to 5; “n” is an integer from 1 to 5; Ar is a C₃-C₄₀ aromatic radical which is free of aliphatic carbon-hydrogen bonds; and X is halogen.

2. The monomer composition according to claim 1, wherein “n” is 1.

3. The monomer composition according to claim 1, wherein “n” is 2.

4. The monomer composition according to claim 1, wherein “m” is 2.

5. The monomer composition according to claim 1, wherein “m” is 3.

6. The monomer composition according to claim 1, wherein Ar is a C₆-C₂₀ aromatic radical.

7. The monomer composition according to claim 1, wherein Ar is a trivalent aromatic radical having structure II

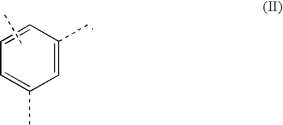

8. The monomer composition according to claim 1, wherein Ar is a trivalent aromatic radical having structure III

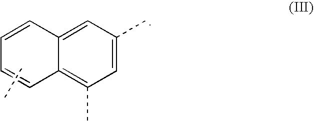

9. The monomer composition according to claim 1, having structure IV

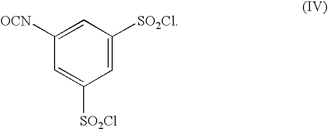

10. The monomer composition according to claim 1, having structure V

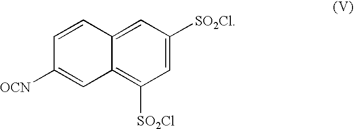

11. The monomer composition according to claim 1, further comprising a C₃-C₄₀ aromatic monomer having a functionality of two.

12. The monomer composition according to claim 1, further comprising a C₃-C₄₀ aromatic halosulfonyl isocyanate having a functionality of two.

13. The monomer composition according to claim 12, wherein the halosulfonyl isocyanate having a functionality of two has structure VI 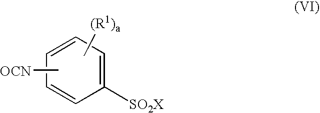
wherein R¹ is independently at each occurrence, a hydrogen atom, a halogen atom, a nitro group, a cyano group, a C₁-C₂₀ aliphatic radical, a C₃-C₂₀ cycloaliphatic radical, or a C₃-C₂₀ aromatic radical; “a” is an integer from 1 to 4; and X is halogen.

14. The monomer composition according to claim 13, wherein the halosulfonyl isocyanate having a functionality of two has structure VI and X is chlorine.

15. The monomer composition according to claim 13, wherein the halosulfonyl isocyanate having a functionality of two has structure VII 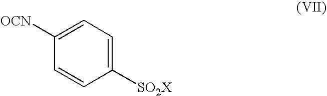
wherein X is halogen.

16. The monomer composition according to claim 13, wherein the halosulfonyl isocyanate having a functionality of two has structure VIII 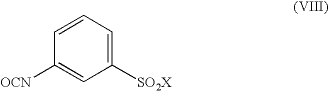
wherein X is halogen.

17. A monomer composition comprising a trivalent aromatic halosulfonyl isocyanate having structure IV 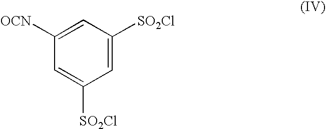
and a halosulfonyl isocyanate having a functionality of two having structure VI 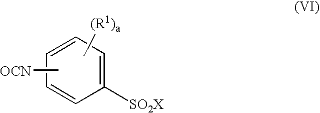
wherein R¹ is independently at each occurrence, a hydrogen atom, a halogen atom, a nitro group, a cyano group, a C₁-C₂₀ aliphatic radical, a C₃-C₂₀ cycloaliphatic radical, or a C₃-C₂₀ aromatic radical; “a” is an integer from 1 to 4; and X is halogen.

18. The monomer composition according to claim 17, wherein the halosulfonyl isocyanate having a functionality of two has structure VII 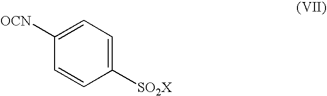
wherein X is halogen.

19. The monomer composition according to claim 17, wherein the halosulfonyl isocyanate having a functionality of two has structure VIII 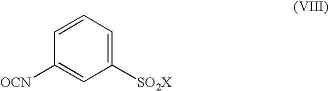
wherein X is halogen.

20. A monomer composition comprising a trivalent aromatic halosulfonyl isocyanate having structure V 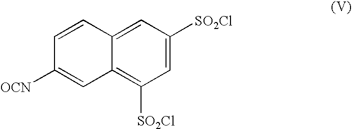
and a halosulfonyl isocyanate having a functionality of two having structure VI 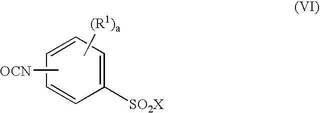
wherein R¹ is independently at each occurrence, a hydrogen atom, a halogen atom, a nitro group, a cyano group, a C₁-C₂₀ aliphatic radical, a C₃-C₂₀ cycloaliphatic radical, or a C₃-C₂₀ aromatic radical; “a” is an integer from 1 to 4; and X is halogen.

21. The monomer composition according to claim 20, wherein the halosulfonyl isocyanate having a functionality of two has structure VII 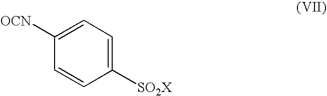
wherein X is halogen.

22. The monomer composition according to claim 20, wherein the halosulfonyl isocyanate having a functionality of two has structure VIII 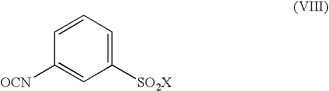
wherein X is halogen.
